(19) United States
(12) Patent Application Publication
(10) Pub. No.: US 2009/0259185 A1
(43) Pub. Date: Oct. 15, 2009

(54) SELF-CONFORMING SURGICAL SEAL

(75) Inventor: Gregory G. Okoniewski, North Haven, CT (US)

Correspondence Address: Tyco Healthcare Group LP, 60 MIDDLETOWN AVENUE, NORTH HAVEN, CT, 06473, US

(73) Assignee: Tyco Healthcare Group LP

(21) Appl. No.: 12/400,842

(22) Filed: Mar. 10, 2009

Related U.S. Application Data
(60) Provisional application No. 61/044,955, filed on Apr. 15, 2008.

(51) Int. Cl. A61B 17/34

(52) U.S. Cl. 604/167.01

(57) ABSTRACT

A surgical access apparatus includes an access member defining a longitudinal axis and having a longitudinal passageway for reception and passage of a surgical object, a seal member mounted to the access member and having inner seal portions defining an aperture to removably receive the surgical object in substantial fluid-tight sealing relation therewith and at least one cable member. The at least one cable member has a first cable end connected to a first radial section of the seal member and a second cable end connected to a second radial section of the seal member displaced from the first radial section. The first and second cable ends are adapted to be laterally displaced relative to the longitudinal axis during offset lateral movement of the surgical object, to thereby cause corresponding lateral displacement of the inner seal portions of the seal member whereby the inner seal portions maintain the substantial fluid tight sealing relation with the surgical object.

### CROSS REFERENCE TO RELATED APPLICATION

The present application claims the benefit of and priority to U.S. Provisional Application Ser. No. 61/044,955, filed on Apr. 15, 2008, the entire contents of which are incorporated herein by reference.

### BACKGROUND

1. Technical Field

The present disclosure relates to a surgical seal of the type adapted for the sealed reception of a surgical object. In particular, this disclosure relates to a surgical seal for use with a surgical access member such as a cannula or trocar assembly.

2. Background of the Related Art

Many contemporary medical and surgical procedures are performed through access members. These devices incorporate narrow tubes or cannulas percutaneously inserted into a patient's body and have a central opening through which surgical objects are introduced and manipulated during the course of the procedure. Generally, such procedures are referred to as “endoscopic”, and, if performed on the patient's abdomen, the procedure is referred to as “laparoscopic”. Throughout the present disclosure, the term “minimally invasive” should be understood to encompass both endoscopic and laparoscopic procedures.

Generally, during minimally invasive procedures, prior to the introduction of a surgical object into the patient's body, insufflation gases are used to enlarge the area surrounding the target surgical site to create a larger, more accessible workspace. Accordingly, the maintenance of a substantially fluid-tight seal along the central opening of the access member, in both the presence and absence of a surgical object, is desirable so as to prevent the escape of the insufflation gases and the deflation or collapse of the enlarged surgical workspace. To this end, surgical access members generally incorporate one or more seals many varieties of which are known in the art. One such example may be seen in commonly assigned U.S. Pat. No. 5,512,053 to Pearson, the entire contents of which are hereby incorporated by reference.

During the course of a minimally invasive procedure, a clinician will frequently move surgical objects laterally within the access member, and the seal disposed therein, to access different regions of the surgical workspace. This lateral movement may distort the seal, thereby potentially causing the escape of insufflation gas and compromising the integrity of the insufflated workspace.

While many varieties of surgical seals are known in the art, a continuing need exists for a seal capable of maintaining the integrity of an insufflated workspace during lateral movement of a surgical object inserted therethrough.

### SUMMARY

Accordingly, the present disclosure is directed to a surgical access apparatus, including an access member defining a longitudinal axis and having a longitudinal passageway for reception and passage of a surgical object, a seal member mounted to the access member and having inner seal portions defining an aperture to removably receive the surgical object in substantial fluid-tight sealing relation therewith and at least one cable member. The at least one cable member has a first cable end connected to a first radial section of the seal member and a second cable end connected to a second radial section of the seal member displaced from the first radial section. The first and second cable ends are adapted to be laterally displaced relative to the longitudinal axis during offset lateral movement of the surgical object, to thereby cause corresponding lateral displacement of the inner seal portions of the seal member whereby the inner seal portions maintain the substantial fluid tight sealing relation with the surgical object. The first and second cable ends may be connected to respective first and second radial sections of the seal member at locations adjacent the inner seal portions. The first and second radial sections may be in general diametrically opposed relation. First and second cable members may be provided with each cable member having cable ends connected to spaced radial sections of the seal member.

The first cable end may be connected to the first radial section at a first location and the second cable end may be connected to the second radial section at a second location with the first and second locations being substantially adjacent the aperture.

The seal member may define at least one channel configured to at least partially receive the at least one cable member. The at least one channel may be defined within the seal member. Alternatively, the at least one channel is defined on an outer surface of the seal member.

The at least one cable member may define a length that remains substantially constant during use of the surgical seal member. The at least one cable member may be formed of a substantially non-rigid material.

The seal member may be formed of an elastomeric material such that the seal member resiliently transitions between first and second conditions upon the respective insertion and removal of the surgical object. The aperture of the seal member may define a first diameter in the first condition and a second diameter in the second condition. The at least one cable member is configured to displace the aperture of the seal member upon lateral manipulation of the surgical object inserted therethrough with the aperture being displaced in the direction of lateral manipulation such that the second diameter of the aperture remains substantially constant.

The access member may include a cannula defining a longitudinal opening dimensioned for passage of the surgical object. A housing may be coupled to the cannula. The housing is configured to receive the seal member and defines at least one cable conduit. The at least one cable conduit is configured to permit displacement of the at least one cable member during lateral manipulation of the surgical object within the seal member.

These and other features of the surgical seal disclosed herein will become more readily apparent to those skilled in the art from the following detailed description of various embodiments of the present disclosure.

### BRIEF DESCRIPTION OF THE DRAWINGS

Various embodiments of the present disclosure are described hereinbelow with references to the drawings, wherein:

FIG. 1A is a top perspective view of a seal member in accordance with the principles of the present disclosure.

FIG. 1B is a top perspective view of one embodiment of the seal member of FIG. 1A.

FIG. 2A is a top plan view of the seal member of FIG. 1A shown in a first condition.

FIG. 2B is a top perspective view of the seal member of FIG. 1A shown in a second condition with a surgical object inserted therethrough.

FIG. 2C is a top plan view of the seal member of FIG. 1A with first and second cable members.

FIG. 3A is a top perspective view of another embodiment of the seal member of FIG. 1 including channels defined in the proximal surface thereof.

FIG. 3B is a side plan view of another embodiment of the seal member of FIG. 1 including a channel defined in the periphery thereof.

FIG. 3C is a top plan view of another embodiment of the seal member of FIG. 1 including channels defined within the seal member.

FIG. 3D is a side plan view of the seal member of FIG. 3C;

FIG. 4A is a perspective view with parts separated of a surgical access member for use in conjunction with the seal member of FIG. 1.

FIG. 4B is side cross-sectional view of the housing of the surgical access member taken along line 4B-4B of FIG. 4A.

FIG. 5 is a top perspective view of the seal member of FIG. 1 illustrating the forces exerted thereupon by a surgical object following insertion.

FIG. 6 is a top plan view of a known, exemplary seal member upon lateral manipulation of a surgical object inserted therethrough.

FIG. 7 is a top plan view of the seal member of FIG. 5 upon lateral manipulation of the surgical object.

### DETAILED DESCRIPTION OF THE EMBODIMENTS

In the drawings and in the description which follows, in which like reference numerals identify similar or identical elements, the term “proximal” will refer to the end of the apparatus closest to a clinician during the use thereof, while the term “distal” will refer to the end which is furthest from the clinician, as is traditional and known in the art.

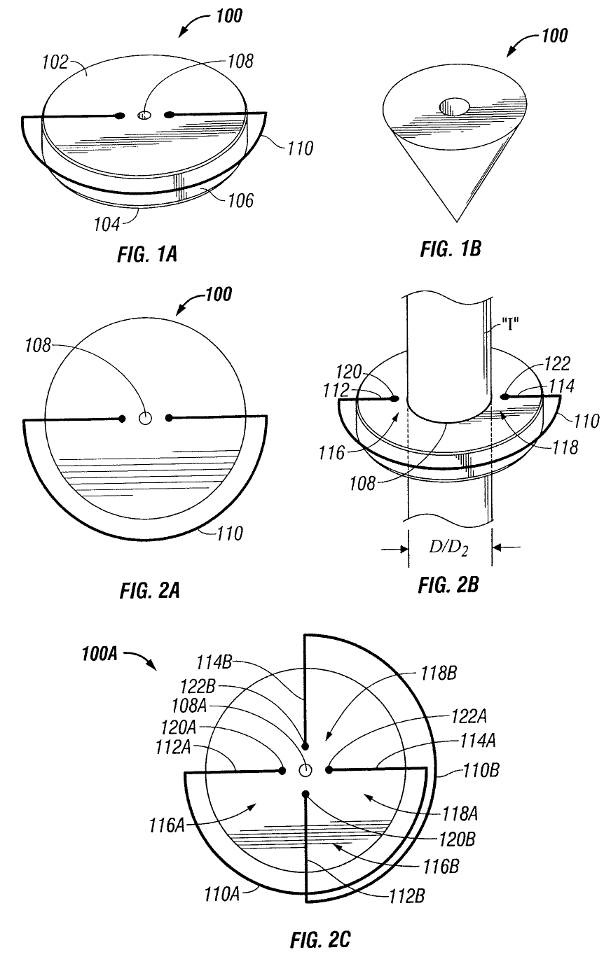

With reference to FIGS. 1A-1B, a seal member 100 is disclosed that defines respective proximal and distal surfaces 102, 104, a periphery 106 and an aperture 108 that is configured to removably receive a surgical object “I” (FIG. 2B) such that a substantially fluid-tight seal is formed therewith. Seal member 100 includes at least one cable member 110 which is discussed in detail below.

Seal member 100 may exhibit any configuration suitable for the intended purpose of receiving surgical object “I” so as to form a substantially fluid-tight seal therewith, including but not being limited to a substantially planar configuration, as seen in FIGS. 1A, or a generally conical configuration, as seen in FIG. 1B.

Seal member 100 may be formed of any suitable biocompatible material that is at least semi-resilient in nature, including but not limited to elastomeric materials. Forming seal member 100 of such a material facilitates the resilient deformation of seal member 100, and aperture 108 in particular, upon the insertion and removal of surgical object “I”. The resilient nature of seal member 100 allows seal member 100 to exhibit various degrees of deformation during use, thereby facilitating the accommodation of surgical objects of various sizes, as well as the maintenance of a substantially fluid-tight seal therewith during the axial or lateral manipulation thereof within seal member 100, as discussed in further detail below.

As seen in FIGS. 2A-2B, prior to receiving surgical object “I”, seal member 100 is in a first condition in which aperture 108 of seal member 100 defines a first diameter “D₁” that is substantially less than the diameter “D” of surgical object “I”. Aperture 108 may be closed in the first position, i.e., such that “D₁” equals zero, to thereby prevent the escape of any insufflation gas through seal member 100 in the absence of surgical object “I”. Upon the insertion of surgical object “I”, aperture 108 deforms, or stretches, to accommodate the larger diameter “D” of surgical object “I”, thereby transitioning into a second condition. In the second condition, aperture 108 of seal member 100 defines a second diameter “D₂” that substantially approximates the diameter “D” of surgical object “I”, thereby forming a substantially fluid-tight seal with surgical object “I” and substantially preventing the escape of insufflation gas. The diameter “D” of the surgical object “I”, and thus the diameter “D₂” of the aperture 108 of seal member 100 in the second condition, will generally lie within the range of about 5 mm to about 15 mm, as is conventional in the art, although substantially greater and lesser values for diameter “D₂” are also within the scope of the present disclosure.

Referring still to FIGS. 2A-2B, cable member 110 of seal member 100 will be discussed.

The cable member, or members, 110 may be formed of any suitable biocompatible material that is substantially non-rigid and substantially non-extensible in character, e.g. stainless steel, polymeric material, etc., such that the length of cable member 110 remains substantially constant during the use of seal member 100. Cable member 110 has respective first and second ends 112, 114 that are attached to seal member 100 at respective first and second sections 116, 118 thereof. The first and second ends 112, 114 of cable member 110 are attached to the first and second sections 116, 118 at first and second locations 120, 122, respectively, that are disposed substantially adjacent to aperture 108 and spaced apart from one another. Various arrangements for securing the first and second ends 112, 114 of the cable member 110 are envisioned. For example, seal member 100 may have posts embedded within the material of the seal member 100. The first and second ends 112, 114 may be attached or secured to the posts. In the alternative, the first and second ends 112, 114 may be embedded within the seal member 100 during manufacture of the seal member 100, such as, for example, during a molding process. Other means for attaching the first and second ends 112, 114 are also envisioned. The respective first and second sections 116, 118, and consequently the respective first and second locations 120, 122, are in substantially diametric opposition to each other.

With reference now to FIG. 2C, in one embodiment, a seal member 100_{A} is disclosed that includes a first cable member 110_{A} having respective first and second ends 112_{A}, 114_{A} attached to first and second sections 116_{A}, 118_{A} of seal member 100_{A} at first and second locations 120_{A}, 122_{A}, respectively. In this embodiment, seal member 100_{A} further includes a second cable member 110_{B} having respective first and second ends 112_{B}, 114_{B} attached to first and second sections 116_{B}, 118_{B} of seal member 100_{A} at first and second locations 120_{B}, 122_{B}, respectively. As with seal member 100 of FIGS. 2A-2B, each of the first and second locations 120_{A}, 122_{A}, 120_{B}, 122_{B} are disposed substantially adjacent aperture 108_{A} and spaced apart from one another. The incorporation of additional cable members, e.g., a second cable member 110_{B}, or three or more cable members, facilitates more uniform deformation of aperture 108_{A} upon laterally manipulating a surgical object “I” (FIG. 2B) inserted therethrough, as discussed below.

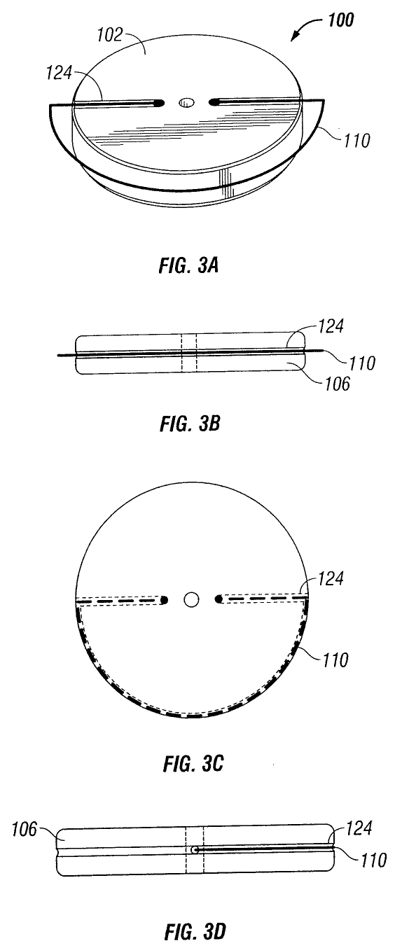

As seen in FIGS. 3A-3D, in one embodiment, seal member 100 defines at least one channel 124 configured to at least partially receive cable member 110. Channels 124 may be formed either in an outer surface of seal member 100, e.g. proximal surface 102 (FIG. 3A), distal surface 104 (not shown) or periphery 106 (FIG. 3B), or within seal member 100 (FIGS. 3C-3D) such that cable member 110 is at least partially concealed by seal member 100.

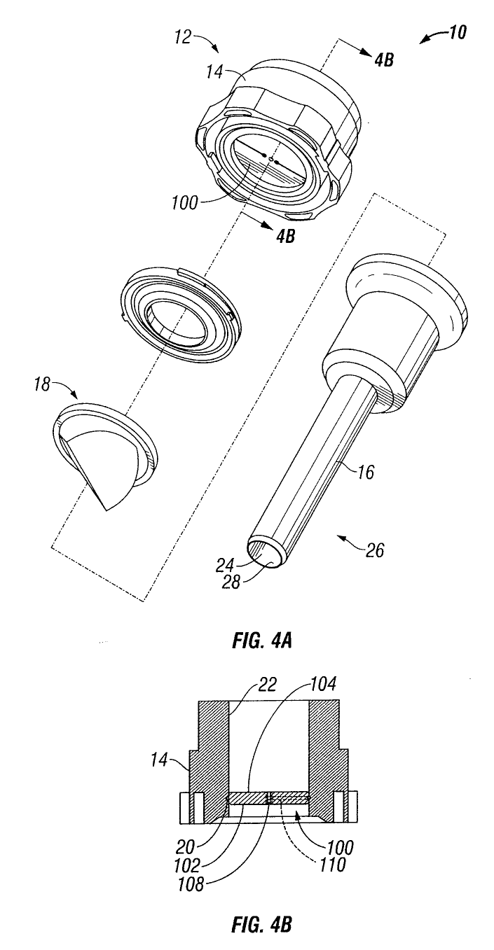

Referring now to FIG. 4A, a surgical access member, in the form of, e.g., a cannula assembly 10, is illustrated that may be used in conjunction with seal member 100. At a proximal end 12, cannula assembly 10 includes a housing 14 that is configured to accommodate the seal 100 that is the subject of the present disclosure. Extending distally from housing 14 is a cannula or elongate member 16. As illustrated, cannula assembly 10 may optionally further include a zero-closure valve 18.

Housing 14 may be any structure suitable for the intended purpose of accommodating seal member 100. As seen in FIG. 4B, in one embodiment, housing 14 defines at least one conduit 20 on an internal surface 22 thereof. Conduit 20 is configured to receive cable member 110 and to permit the displacement thereof during lateral manipulation of surgical object “I” within seal member 100, as discussed in further detail below. Further information regarding seal housing 14 may be obtained through reference to commonly owned U.S. Pat. No. 7,169,130 to Exline et al., the entire contents of which are hereby incorporated by reference.

Cannula 16 extends distally from housing 14 and defines a longitudinal passage 24 that is configured to permit a surgical object “I” (FIG. 2B), to pass therethrough, e.g., an obturator, trocar or endoscope. At its distal end 26, cannula 16 defines an opening 28 that is configured to allow the surgical object “I” to pass therethrough. Conventionally, surgical objects generally define a diameter substantially within the range of about 3 mm to about 15 mm. Accordingly, longitudinal passage 24 will be dimensioned similarly, although substantially larger and smaller surgical objects and a cannula 16 defining a substantially larger or smaller longitudinal passage 24 and opening 28 are also within the scope of the present disclosure.

Referring now to FIGS. 2A, 4A and 5-7, the use and function of seal member 100 will be described in conjunction with a surgical access apparatus, e.g., cannula assembly 10. Initially, the target surgical site is insufflated with a suitable biocompatible gas, e.g., CO₂ gas, such that a larger internal workspace may be created within a patient, thereby providing greater access to the patient's internal organs and/or cavities. The insufflation may be performed with an insufflation needle or similar device, as is conventional in the art. Thereafter, a variety of surgical objects, depicted generally as surgical object “I”, are inserted into cannula assembly 10 and advanced distally through seal member 100 and elongate member 16 to percutaneously access the insufflated workspace and carryout the minimally invasive procedure.

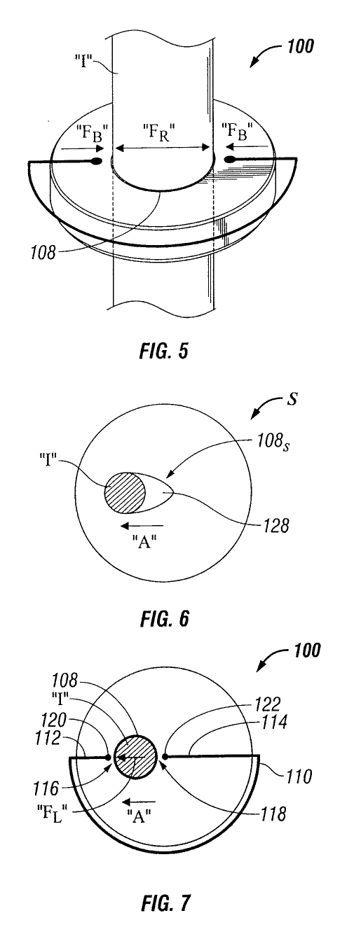

Subsequent to insufflation, seal member 100 substantially prevents the escape of insufflation gas, thereby maintaining the integrity of the insufflated workspace in both the absence and presence of surgical object “I”. As seen in FIG. 2A, prior to the insertion of surgical object “I”, seal member 100 is in the first condition, in which aperture 108 defines a first diameter “D₁”. Upon the insertion of surgical object “I” (FIG. 5), seal member 100, and in particular the aperture 108 thereof, is subjected to a force “F_{R}” applied by surgical object “I” that is directed radially outward. Force “F_{R}” forces open aperture 108, thereby transitioning seal member 100 into the second condition thereof in which aperture 108 defines a second, larger diameter “D₂” that substantially approximates the diameter “D” of surgical object “I”. In the second condition, aperture 108 exerts a biasing force “F_{B}” directed radially inward that attempts to return seal member 100 to the first condition. Biasing force “F_{B}” is exerted upon surgical object “I”, thereby creating a substantially fluid-tight seal therewith.

As previously discussed, it is often necessary to axially or laterally manipulate surgical object during the course of a minimally invasive procedure to access different areas of a surgical workspace. FIG. 6 describes the impact of such lateral manipulation upon a known seal “S”. As would be appreciated by one of ordinary skill, laterally manipulating surgical object “I” in the direction of arrow “A” can laterally distort the enlarged aperture 108s of the seal “S”, thereby creating a leak path 128 and potentially resulting in the escape insufflation gas therethrough. Seal member 100 of the present disclosure mitigates this potentiality through the incorporation cable member, or members, 110.

As seen in FIG. 7, upon the lateral movement of surgical object “I” in the direction of arrow “A”, a force “F_{L}” is applied to seal member 100 at the first section 120 thereof. Force “F_{L}” attempts to distort aperture 108 in the direction of arrow “A” and thereby create a leak path 128 (FIG. 6). Upon the application of force “F_{L}” to the first section 116, the first end 112 of cable member 110 is subjected to force “F_{L}” through the connection between the first end 112 of cable member 110 and seal member 100 at first location 120. Force “F_{L}” displaces the first section 116, thereby displacing the first end 112 of cable member 110, and ultimately the second end 114 thereof. Through the connection between the second end 114 of cable member 110 and seal member 100 at the second location 122, the second section 118 of seal member 100 is subjected to the influence of force “F_{L}” and is also displaced in the direction indicated by arrow “A”. Consequently, aperture 108 deforms in a substantially uniform manner, maintaining its diameter D₂ in the second condition and minimizing the dimensions of leak path 128, if any, such that the substantially fluid-tight seal formed with surgical object “I” is preserved and the escape of insufflation gas through seal member 100 is curtailed. The incorporation of additional cable members (FIG. 2C) further ensures uniform deformation of aperture 108 upon the lateral movement of surgical object “I” and the preservation of a substantially fluid-tight seal therewith. The present disclosure contemplates that that material comprising cable member 110, and the configuration and dimensions thereof, may be such that the degree of distortion realized by the second section 118 of the seal member 100 will approximate that of the first section 116, thereby substantially maintaining the diameter “D₂” of aperture 108 in the second condition during the lateral manipulation of surgical object “I”.

Although the illustrative embodiments of the present disclosure have been described herein with reference to the accompanying drawings, the above description, disclosure, and figures should not be construed as limiting, but merely as exemplifications of particular embodiments. It is to be understood, therefore, that the disclosure is not limited to those precise embodiments, and that various other changes and modifications may be effected therein by one skilled in the art without departing from the scope or spirit of the disclosure.

What is claimed is:

1. A surgical access apparatus, which comprises:
an access member defining a longitudinal axis and having a longitudinal passageway for reception and passage of a surgical object;
a seal member mounted to the access member, the seal member having inner seal portions defining an aperture to removably receive the surgical object in substantial fluid-tight sealing relation therewith; and
at least one cable member, the at least one cable member having a first cable end connected to a first radial section of the seal member and a second cable end connected to a second radial section of the seal member displaced from the first radial section, the first and second cable ends adapted to be laterally displaced relative to the longitudinal axis during offset lateral movement of the surgical object, to thereby cause corresponding lateral displacement of the inner seal portions of the seal member whereby the inner seal portions maintain the substantial fluid tight sealing relation with the surgical object.

2. The surgical access apparatus of claim 1, wherein the first and second cable ends are connected to respective first and second radial sections of the seal member at locations adjacent the inner seal portions.

3. The surgical access apparatus of claim 2, wherein the first and second radial sections are in general diametrically opposed relation.

4. The surgical access apparatus of claim 2 including first and second cable members, each cable member having cable ends connected to spaced radial sections of the seal member.

5. The surgical access apparatus of claim 1, wherein the first cable end is connected to the first radial section at a first location and the second cable end is connected to the second radial section at a second location, the first and second locations being substantially adjacent the aperture.

6. The surgical access apparatus of claim 1, wherein the seal member defines at least one channel configured to at least partially receive the at least one cable member.

7. The surgical access apparatus of claim 6, wherein the at least one channel is defined within the seal member.

8. The surgical access apparatus of claim 6, wherein the at least one channel is defined on an outer surface of the seal member.

9. The surgical access apparatus of claim 1, wherein the at least one cable member defines a length that remains substantially constant during use of the surgical seal member.

10. The surgical access apparatus of claim 1, wherein the at least one cable member is formed of a substantially non-rigid material.

11. The surgical access apparatus of claim 1, wherein the seal member is formed of an elastomeric material such that the seal member resiliently transitions between first and second conditions upon the respective insertion and removal of the surgical object

12. The surgical access apparatus of claim 11, wherein the aperture of the seal member defines a first diameter in the first condition and a second diameter in the second condition.

13. The surgical access apparatus of claim 12, wherein the at least one cable member is configured to displace the aperture of the seal member upon lateral manipulation of the surgical object inserted therethrough, the aperture being displaced in the direction of lateral manipulation such that the second diameter of the aperture remains substantially constant.

14. The surgical access apparatus according to claim 1, wherein the access member includes a cannula defining a longitudinal opening dimensioned for passage of the surgical object.

15. The surgical access apparatus according to claim 14, further including a housing coupled to the cannula, the housing being configured to receive the seal member and defining at least one cable conduit, the at least one cable conduit being configured to permit displacement of the at least one cable member during lateral manipulation of the surgical object within the seal member.
